(19) United States
(12) Patent Application Publication
(10) Pub. No.: US 2014/0276062 A1
(43) Pub. Date: Sep. 18, 2014

(54) ULTRASOUND DIAGNOSTIC DEVICE AND METHOD FOR CONTROLLING ULTRASOUND DIAGNOSTIC DEVICE

(71) Applicant: KONICA MINOLTA, INC., Chiyoda-ku, Tokyo (JP)

(72) Inventor: Youichi Kondoh, Tokyo (JP)

(21) Appl. No.: 14/349,655

(22) PCT Filed: Oct. 5, 2012
(86) PCT No.: PCT/JP2012/006413

§ 371 Date: Apr. 3, 2014

(30) Foreign Application Priority Data
Oct. 7, 2011 (JP) 2011-222485

(51) Int. Cl. A61B 8/00; A61B 8/14; A61B 8/08

(52) U.S. Cl. CPC A61B 8/54; A61B 8/0891; A61B 8/5223; A61B 8/463; A61B 8/5207; A61B 8/14
USPC 600/443

(57) ABSTRACT

An ultrasound diagnostic device to which an ultrasound probe is connectable, measuring an IMT of a vascular wall in a carotid artery, includes: a transmission-reception processing unit supplying a transmission signal causing the ultrasound probe to transmit an ultrasound to short-axis cross-sections along the carotid artery, and receiving a signal based on a reflected ultrasound and generating a reception signal for frames at each position; a two-dimensional image generation unit generating two-dimensional images corresponding to the frames; a bulb boundary detection unit measuring a perimeter or area of the vascular wall in each image, and detecting a boundary between a CCA and a CCA bulb from the perimeter or area; and position information indicating a position where each image is acquired; a ROI determination unit determining a ROI defining an IMT measurement range with respect to the boundary; and an IMT measurement unit measuring the IMT within the images.

### TECHNICAL FIELD

The present disclosure pertains to an ultrasound diagnostic device and to a control method for the ultrasound diagnostic device, and particular pertains to carotid artery diagnostic technology for early discovery of arteriosclerosis.

### BACKGROUND ART

In recent years, increasing numbers of patients are suffering from circulatory problems, including ischemia, cerebral infarctions, and myocardial infarction. Early discovery and treatments of arteriosclerosis indicators is important for preventing the above problems.

One indicator for diagnosing arteriosclerosis is a thickness measurement of the tunica intima-tunica media complex in the carotid artery (hereinafter also IMT, Intima-Media Thickness). The IMT is an important index for initial detection of atherosclerosis in the carotid artery.

FIG. 14 is a cross-sectional diagram of a cross-section taken along a longitudinal direction (i.e., the direction of arterial extension) of a blood vessel, which is the carotid artery (hereinafter also termed a longitudinal cross-section). The blood vessel includes a vascular wall 201 and a lumen 204. The vascular wall 201 is, in turn, made up of a tunica intima 202, a tunica media 203, and a tunica adventitia 205, as ordered from innermost to outermost. Also, an intima-media 206 is a complex of the tunica intima 202 and the tunica media 203. The thickness of the intima-media 206 is the IMT. The ultrasound diagnostic device is usable for identifying the intima-media 206 between the lumen 204 and the tunica adventitia 205.

Ultrasound exams, being simple and non-invasive, are used for IMT measurement. IMT measurement is performed on the carotid artery because the carotid artery is a predilection site for arteriosclerosis, and because measurement of the carotid artery is easily performed with ultrasounds at a comparatively shallow depth of 2 cm to 3 cm from the skin surface. Ordinarily, IMT measurement is performed on a two-dimensional image obtained as an ultrasound diagnostic image of a cross-section taken along the longitudinal direction of the blood vessel. Specifically, as shown in FIG. 14, IMT measurement is performable by detecting a boundary between the lumen 204 and the tunica intima 202 (hereinafter also termed lumen-intima boundary 207) and a boundary between the tunica media 203 and the tunica adventitia 205 (hereinafter also termed media-adventitia boundary 208).

FIG. 15 is a perspective view diagram of a carotid artery taken along the longitudinal direction thereof. As shown, the carotid artery is made up of a Common Carotid Artery (hereinafter also CCA) 213 arranged in the central direction, as well as an Internal Carotid Artery (hereinafter also ICA) 215 and an External Carotid Artery (hereinafter also ECA) 216 arranged in the peripheral direction. The bulb of the common carotid artery (hereinafter simply termed a bulb) 214 is located between CCA 213 and the ICA 215 and ECA 216. The bifurcation of the common carotid artery (hereinafter also Bif) 217 is found at the point where the ICA 215 and ECA 216 split off from the bulb 214. The range for measuring the IMT may be, for example, defined as starting from the boundary 219 between the CCA 213 and the bulb 214 (hereinafter also CCA-bulb boundary 219) and extending along a later-described far wall toward the CCA 213 for a range of 1 cm, such as the recommended IMT measurement range 212 described in Non-Patent Literature 1.

FIG. 16 is a schematic diagram of a two-dimensional image taken along the longitudinal cross-section of the carotid artery. The ultrasound diagnostic device performs measurement in the IMT measurement range 212 by defining a Region of Interest 211 (hereinafter also ROI 211) so as to intersect both of a vascular wall relatively far from the skin surface (hereinafter, back wall 209) and a vascular wall relatively close to the skin surface (hereinafter, front wall 210), as shown in FIG. 16. The IMT measurement range 212 is then defined within the ROI 211 on the vascular wall. An IMT value is calculated as the maximum thickness (hereinafter also maxIMT) or mean thickness (hereinafter also mean IMT) of the IMT within the IMT measurement range 212.

Operations for determining the ROI 211 that defines the IMT measurement range 212 are complex when these operations must be performed manually. As such, technology has been proposed for simplifying these complex operations and enabling simpler IMT measurement, such as Patent Literature 1 and Patent Literature 2, in which ROI determination is automatized. For instance, in Patent Literature 1, intensity values are added and averaged for each pixel in the two-dimensional image of a blood vessel longitudinal cross-section obtained by transmitting and receiving an ultrasound beam. Vascular wall positions are then extracted by using an inflection point of intensity values in the ultrasound beam transmission direction, and the ultrasound diagnostic device detects the ROI in the two-dimensional image. Also, Patent Literature 2 discloses a ultrasound diagnostic device that determines the ROI by detecting a cardiac wall two-dimensionally by binarizing a brightness signal of a cardiac wall two-dimensional image.

### CITATION LIST

### Patent Literature

### [Patent Literature 1]

Japanese Patent Application Publication No. 2010-119842

### [Patent Literature 2]

Japanese Patent Application Publication No. 2002-125971

### Non-Patent Literature

### [Non-Patent Literature 1]

Journal of the American Society of Echocardiography, February 2008, pages 93-111

### [Non-Patent Literature 2]

Research Group for Early Arteriosclerosis, “maxIMT Measurement”, [online], Sep. 9, 2010 (retrieved Oct. 5, 2011), URL: http://www.imt-ca.com/contents/e08.html

### SUMMARY OF INVENTION

### Technical Problem

However, Patent Literature 1 and Patent Literature 2 describe technology for determining the ROI as crossing the vascular wall, and do not automatically determine the ROI defining the measurement range for IMT measurement in the longitudinal direction of the vascular wall. However, with these methods, an operator must determine the ROI for the carotid artery in the longitudinal direction. As a result, measurements are difficult for an inexperienced operator to make, and the diagnostic time needed for precise measurement is longer.

In consideration of these concerns, an ultrasound diagnostic device pertaining to an aspect of the disclosure enables automatic determination of an ROI defining a measurement range for measuring the IMT of a carotid artery vascular wall. The present disclosure aims to provide an ultrasound diagnostic device and ultrasound diagnostic device control method enabling swift IMT measurement through a simple operation performable by an inexperienced operator.

### Solution to Problem

In order to achieve this aim, an ultrasound diagnostic device to which an ultrasound probe is connectable, measuring an IMT of a vascular wall in a carotid artery, comprises: a transmission and reception processing unit performing a transmission process of supplying a transmission signal for causing the ultrasound probe to transmit an ultrasound to a short-axis cross-section at a plurality of different positions along the carotid artery, and a reception process of receiving a signal based on a reflected ultrasound received by the ultrasound probe from the carotid artery and generating a reception signal for a plurality of frames each corresponding to the short-axis cross-section at one of the different positions; a two-dimensional image generation unit generating a plurality of two-dimensional images each corresponding to one of the frames, based on the reception signal for each of the frames; a bulb boundary detection unit measuring one or more of a perimeter and a cross-sectional area of the vascular wall in the carotid artery according to the short-axis cross-section in each of the two-dimensional images, and detecting a boundary between a common carotid artery and a common carotid artery bulb according to: at least one of the perimeter and the cross-sectional area; and position information indicating a respective position along the carotid artery where each of the two-dimensional images is acquired; a ROI determination unit determining a ROI that defines a measurement range for measuring the IMT, with respect to the boundary; and an IMT measurement unit measuring the IMT in at least one of the two-dimensional images in the ROI.

Also, a control method for an ultrasound diagnostic device to which an ultrasound probe is connectable, measuring an IMT of a vascular wall in a carotid artery comprises: supplying a transmission signal for causing the ultrasound probe to transmit an ultrasound to a short-axis cross-section at a plurality of different positions along the carotid artery; receiving a signal based on a reflected ultrasound received by the ultrasound probe from the carotid artery and generating a reception signal for a plurality of frames each corresponding to the short-axis cross-section at one of the different positions; generating a plurality of two-dimensional images each corresponding to one of the frames, based on the reception signal for each of the frames; measuring one or more of a perimeter and a cross-sectional area of the vascular wall in the carotid artery according to the short-axis cross-section in each of the two-dimensional images, and detecting a boundary between a common carotid artery and a common carotid artery bulb according to: at least one of the perimeter and the cross-sectional area; and position information indicating a respective position along the carotid artery where each of the two-dimensional images is acquired; determining a ROI that defines a measurement range for measuring a predetermined IMT, with respect to the boundary; and measuring the IMT in the ROI within each of the two-dimensional images.

### Effects of Invention

An ultrasound diagnostic device pertaining to an aspect of the disclosure enables automatic determination of an ROI defining a measurement range for measuring the IMT of a carotid artery vascular wall. The present disclosure aims to provide an ultrasound diagnostic device and ultrasound diagnostic device control method enabling swift IMT measurement through a simple operation performable by an inexperienced operator.

### BRIEF DESCRIPTION OF DRAWINGS

FIG. 1 is a block diagram illustrating the configuration of an ultrasound diagnostic device 1 pertaining to Embodiment 1.

FIG. 2 is a schematic diagram representing a hand scan performed in a longitudinal direction of a carotid artery using the ultrasound probe 2 of the ultrasound diagnostic device 1 pertaining to Embodiment 1.

FIG. 3 is a block diagram illustrating the configuration of a cross-section information analysis unit 8 in the ultrasound diagnostic device 1 pertaining to Embodiment 1.

FIG. 4 is a flowchart of operations pertaining to IMT measurement by the ultrasound diagnostic device 1 of Embodiment 1.

FIGS. 5A-5C illustrate data based on a two-dimensional image indicating a short-axis cross section of the carotid artery as obtained by the ultrasound diagnostic device 1 of Embodiment 1, where FIG. 5A illustrates a two-dimensional image 100, FIG. 5B illustrates a carotid artery border 101 in each frame, and FIG. 5C illustrates a three-dimensional image 102 of the carotid artery.

FIG. 6 is a flowchart of the operations performed by the ultrasound diagnostic device 1 of Embodiment 1 to determine whether or not the hand scan has been correctly performed.

FIG. 7A schematically illustrates the carotid artery when the hand scan is performed forward (in the arrow direction), and FIG. 7B schematically shows two-dimensional images as cross-sectional diagrams (A) through (D) of the carotid artery as obtained by the scan of FIG. 7A.

FIG. 8 lists frame numbers in the reception signal obtained by the ultrasound diagnostic device 1 of Embodiment 1, and a perimeter and cross-sectional area for the border 101 in each frame.

FIG. 9 lists frame numbers in the reception signal obtained by the ultrasound diagnostic device 1 of Embodiment 1, and shows a plotted relationship between the frame numbers and the perimeter and cross-sectional area of the border 101 in each frame, in hand scan order.

FIG. 10 is a plotted relationship between the frame numbers and results of taking a moving average from the last two instances and computing the second derivative in each frame, in hand scan order, from the perimeter and cross-sectional area of the border 101 in each frame acquired from the reception signal obtained by the ultrasound diagnostic device 1 of Embodiment 1.

FIG. 11 is a block diagram illustrating the configuration of an ultrasound diagnostic device 1′ pertaining to a variation of Embodiment 1.

FIG. 12 is a perspective view diagram illustrating the configuration of an ultrasound diagnostic device 1A pertaining to Embodiment 2.

FIG. 13 is a block diagram illustrating the configuration of the ultrasound diagnostic device 1A pertaining to Embodiment 1.

FIG. 14 is a cross-sectional diagram of a cross-section taken along a longitudinal direction of the carotid artery.

FIG. 15 is a perspective view diagram of a carotid artery taken along the longitudinal direction thereof.

FIG. 16 is a schematic diagram of a two-dimensional image taken along the longitudinal cross-section of the carotid artery.

FIGS. 17A and 17B are cross-sectional diagrams illustrating the positional relationship of the ultrasound probe and the carotid artery in short-axis cross-section, where the transducer array is aligned with the carotid artery centre in FIG. 17A and is offset from the carotid artery centre in FIG. 17B.

FIG. 18A is a perspective view of the carotid artery along the longitudinal direction, and FIG. 18B is a schematic view of expansion in the vicinity of the CCA-bulb boundary 219 at point (A) in FIG. 18A.

### DESCRIPTION OF EMBODIMENTS

### Background Leading to Embodiments

Varied research into ultrasound diagnostic devices has been conducted in order to determine the ROI 211 defining the IMT measurement range. For example, Non-Patent Literature 2 describes a CCA-bulb boundary 219 detection method. There, the CCA-bulb boundary 219 is determined by representing the CCA-bulb boundary 14 as the inflection point of the vascular wall where the peripheral end of the CCA morphs into the bulb. The inflection point is then used to find an intersection of lines respectively extending from the CCA side and the bulb side of the boundary between the tunica adventitia and tunica media in the vicinity of the transition from CCA to bulb, and that intersection is identified as the CCA-bulb boundary 219. The inventors have used the CCA-bulb boundary 219 identified using a detection method as described in Non-Patent Literature 2 as a basis for dedicated investigation into the realisation of defining the IMT measurement range. For instance, the inventors debated whether or not the predetermined measurement range recommended in Non-Patent Literature 1 is usable for defining the ROI 211.

However, the method of Non-Patent Literature 2 is not able to detect the CCA-bulb boundary 219 for certain subjects. In these cases, the IMT measurement range cannot be automatically detected, thus requiring that the operator determine the IMT measurement range 212 manually. Accordingly, the CCA-bulb boundary 219 detection described in Non-Patent Literature 2 was thought inapplicable as a method for determining the IMT measurement range 212, even if automated.

The inventors then considered the causes. The method of Non-Patent Literature 2 is able to detect the CCA-bulb boundary 219 when the two-dimensional image is obtained for a carotid artery that approximates an ideal shape. However, when a two-dimensional image for a carotid artery having an idiosyncratic shape is used, the CCA-bulb boundary 219 cannot be detected, and thus the IMT measurement range 212 cannot be determined. For instance, when the subject's carotid artery has no curve in the vascular wall of the CCA-bulb boundary 219, detecting the inflection point becomes difficult. Also, despite the CCA-bulb boundary 219 being present in the subject's carotid artery, the inflection point is difficult to detect when the shape is difficult to observe using an ultrasound diagnostic device, and when the curvature of the neck during observation is such that the inflection point of the CCA-bulb boundary 219 cannot be observed. As a result, the CCA-bulb boundary 219 cannot be detected. For example, the above applies when at least one of the front wall and back wall of the bulb is flat, such that the inflection point of the CCA-bulb boundary 219 along the vascular wall is difficult to detect along said wall. Also, when a two-dimensional image is obtained in which the inflection point is difficult to observe, further operations must be repeatedly performed in order to obtain a two-dimensional image in which the inflection point is visible. This has been a problem in diagnosis when the inflection point remains ultimately undetectable.

In order for the ultrasound diagnostic device measuring the IMT of the vascular wall in the carotid artery to automatically determine the ROI 211 defining the measurement range of the IMT, the CCA-bulb boundary 219 must be detected regardless of the subject's carotid artery shape. Establishing a scanning method enabling such detection would be beneficial.

Also, IMT measurement is typically performed with the ultrasound probe arranged at the surface of the neck along the longitudinal direction of the carotid artery. Here, accurate measurement requires that the ultrasound probe be located at a position along the longitudinal direction of the carotid artery so as to intersect the vicinity of the centre in a perpendicular cross section (hereinafter also short-axis cross-section). This caused by the fact that while ultrasounds are reflected by tissue boundaries having different acoustic impedance values, clearer ultrasound echo signals are obtained through strong reflection of an ultrasound perpendicular to the boundary.

FIGS. 17A and 17B are cross-sectional diagrams illustrating the positional relationship of the ultrasound probe and the carotid artery in short-axis cross-section, where the transducer array of the ultrasound probe is aligned with the carotid artery centre in FIG. 17A and is offset from the carotid artery centre in FIG. 17B. As shown in FIG. 17A, the ultrasounds are perpendicular to the lumen-intima boundary 207 and the media-adventitia boundary 208 when the ultrasound probe 2 is placed in the vicinity of the centre of the subject's artery, i.e., when the ultrasound beam travels in a path that intersects the centre 220 of the artery. As such, strong and clear reflections (i.e., ultrasound echo signals) are obtained at both boundaries. Also, as shown in FIG. 17B, when the path of the ultrasound beam does not intersect the centre 220 of the artery, only weak and unclear reflections (i.e., ultrasound echo signals) are obtained as the ultrasound is not perpendicular to the boundaries. As such, the lumen-intima boundary 207 and the media-adventitia boundary 208 are blurry and difficult to extract, and the lumen-intima boundary 207 may not be extractable at all. In such a case, IMT measurement cannot be accurately performed. Furthermore, in some cases meandering arteries may prevent the transducer array from being aligned with the artery centre.

For these reasons, the transducer array must be arranged along the longitudinal direction of the carotid artery in the vicinity of the short-axis cross-section centre in order to realise accurate IMT measurement.

In addition, IMT is normally measured multiple times at regular intervals in a single subject. Accurate diagnosis using such a test requires that the same measurement range be used for each measurement taken.

As such, upon dedicated investigation, the inventors arrived at using a method of detecting the CCA-bulb boundary 219 that does not depend on the condition of the two-dimensional image or on the shape of the subject's carotid artery. FIG. 18A is a perspective view of the carotid artery along the longitudinal direction, and FIG. 18B is a schematic view of expansion in the vicinity of the CCA-bulb boundary 219 at point (A) in FIG. 18A, used in the inventors' considerations. As shown in FIG. 18B, the vascular diameter expands dramatically in the vicinity of the CCA-bulb boundary. The boundary between the CCA and the bulb corresponds to the onset of change in curvature, in terms of the surface shape of the carotid artery along the longitudinal direction. As such, detecting the onset of this change as shown in FIG. 18B has been thought of as a method for extracting the CCA-bulb boundary 219. Furthermore, dedicated investigation was pursued into technology for reducing the effect of the ultrasound probe transducer array being offset from the centre of the carotid artery. As a result, the inventors arrived at the ultrasound diagnostic device of the present disclosure.

An ultrasound diagnostic device and an ultrasound diagnostic device control method pertaining to the Embodiments are described below, with reference to the accompanying drawings.

### Overview of Embodiments

In one aspect, an ultrasound diagnostic device to which an ultrasound probe is connectable, measuring an IMT of a vascular wall in a carotid artery comprises: a transmission and reception processing unit performing a transmission process of supplying a transmission signal for causing the ultrasound probe to transmit an ultrasound to a short-axis cross-section at a plurality of different positions along the carotid artery, and a reception process of receiving a signal based on a reflected ultrasound received by the ultrasound probe from the carotid artery and generating a reception signal for a plurality of frames each corresponding to the short-axis cross-section at one of the different positions; a two-dimensional image generation unit generating a plurality of two-dimensional images each corresponding to one of the frames, based on the reception signal for each of the frames; a bulb boundary detection unit measuring one or more of a perimeter and a cross-sectional area of the vascular wall in the carotid artery according to the short-axis cross-section in each of the two-dimensional images, and detecting a boundary between a common carotid artery and a common carotid artery bulb according to: at least one of the perimeter and the cross-sectional area; and position information indicating a respective position along the carotid artery where each of the two-dimensional images is acquired; a ROI determination unit determining a ROI that defines a measurement range for measuring the IMT, with respect to the boundary; and an IMT measurement unit measuring the IMT in at least one of the two-dimensional images in the ROI.

In another aspect, the bulb boundary detection unit detects the boundary as an onset of change in size of the perimeter or of the cross-sectional area.

In an alternate aspect, the bulb boundary detection unit performs a border extraction process of extracting a border of the vascular wall in the carotid artery from the respective two-dimensional images corresponding to each of the frames, and measures one or more of the perimeter and the cross-sectional area using the border.

In a further aspect, the bulb boundary detection unit performs a determination process of determining whether acquisition of the two-dimensional images has been performed at respective positions that are separated by a predetermined interval with respect to the carotid artery.

In an additional aspect, the bulb boundary detection unit detects a coordinate position of the carotid artery for each of the two-dimensional images, and determines affirmatively when neighbouring two-dimensional images are separated by a gap equal to or less than a predetermined value in terms of the respective coordinate position of the two-dimensional images.

In yet another aspect, the bulb boundary detection unit determines an extension direction of the carotid artery based on the two-dimensional images.

In still another aspect, the bulb boundary detection unit detects a number of cross-sections present in the two-dimensional images and determines the extension direction according to the number of cross-sections and the position information.

In yet a further aspect, the extension direction is one of: a peripheral direction from the common carotid artery toward an internal carotid artery and an external carotid artery; and a central direction from the internal carotid artery and the external carotid artery toward the common carotid artery.

In another alternate aspect, the ROI determination unit determines the ROI for measuring the IMT according to the boundary and the extension direction.

Alternatively, a control method for an ultrasound diagnostic device to which an ultrasound probe is connectable, measuring an IMT of a vascular wall in a carotid artery comprises: supplying a transmission signal for causing the ultrasound probe to transmit an ultrasound to a short-axis cross-section at a plurality of different positions along the carotid artery; receiving a signal based on a reflected ultrasound received by the ultrasound probe from the carotid artery and generating a reception signal for a plurality of frames each corresponding to the short-axis cross-section at one of the different positions; generating a plurality of two-dimensional images each corresponding to one of the frames, based on the reception signal for each of the frames; measuring one or more of a perimeter and a cross-sectional area of the vascular wall in the carotid artery according to the short-axis cross-section in each of the two-dimensional images, and detecting a boundary between a common carotid artery and a common carotid artery bulb according to: at least one of the perimeter and the cross-sectional area; and position information indicating a respective position along the carotid artery where each of the two-dimensional images is acquired; determining a ROI that defines a measurement range for measuring a predetermined IMT, with respect to the boundary; and measuring the IMT in the ROI within each of the two-dimensional images.

The following describes an ultrasound diagnostic device and a control method for the ultrasound diagnostic device as Embodiments pertaining to the present disclosure, with reference to the accompanying drawings.

### Embodiment 1

An ultrasound diagnostic device pertaining to Embodiment 1 is described below, with reference to the accompanying drawings.

### Configuration

(General Configuration)

FIG. 1 is a block diagram illustrating the configuration of an ultrasound diagnostic device 1 pertaining to Embodiment 1.

The ultrasound diagnostic device 1 is configured to be electrically connectable to an ultrasound probe 2 that transmits and receives an ultrasound aimed at a subject. FIG. 1 illustrates the ultrasound diagnostic device 1 as connected to the ultrasound probe 2. The ultrasound diagnostic device 1 includes a controller 300. The controller 300 includes a transmission and reception processing unit 3, a two-dimensional image generation unit 4, a two-dimensional image storage unit 5, a bulb boundary detection unit 15, a ROI detection unit 13, and an IMT measurement unit 14.

(Ultrasound Probe 2)

The ultrasound probe 2 includes a transducer array in which a plurality of piezoelectric elements are arranged into multiple columns. The ultrasound probe 2 converts a transmission signal, which is an electronic signal provided in pulse or continuous form by the later-described transmission and reception processing unit 3, into an ultrasound beam in pulse or continuous form, and when the transducer array is in contact with the skin surface of the test subject, fires the ultrasound beam toward the carotid artery from the skin surface. In order to obtain a two-dimensional image of the carotid artery in short-axis cross-section, the ultrasound probe 2 is arranged such that the transducer array is perpendicular to the longitudinal direction of the carotid artery when the ultrasound beam is fired. The ultrasound probe 2 then receives an ultrasound echo signal, which is an ultrasound reflected from the subject, then converts the ultrasound echo signal into an electronic signal through the transducer array and provides the electronic signal to the transmission and reception processing unit 3.

In order to obtain a plurality of two-dimensional images of the short-axis cross-section of the carotid artery, a hand scan is performed with the transducers arranged at one end of the ultrasound probe 2 being disposed substantially perpendicular to the longitudinal direction of the carotid artery. FIG. 2 is a schematic diagram representing the hand scan performed along the longitudinal direction of the carotid artery using the ultrasound probe 2 of the ultrasound diagnostic device 1 pertaining to Embodiment 1. The transducer array of the ultrasound probe 2 is placed in contact with the skin surface and displaced one way along the longitudinal direction of the carotid artery, and transmits the ultrasound beam. In order to obtain a plurality of two-dimensional images of the short-axis cross-section in a predetermined interval, the displacement of the ultrasound probe 2 is beneficially a motion along the longitudinal direction of the carotid artery performed at a fixed speed. Also, the ultrasound probe 2 is beneficially displaced at a predetermined speed. The spacing between the transducers of the ultrasound probe 2 and the spacing between frames acquired along the displacement direction are beneficially substantially equal. For example, an inter-transducer spacing of 0.25 mm transducer array and an inter-frame spacing of 2.5 mm are beneficial. For instance, when the ultrasound diagnostic device 1 has a frame rate of 20 fps and the ultrasound probe 2 has an inter-transducer spacing of 2.5 mm, then displacing the ultrasound probe 2 at a speed of 4 mm/s to 6 mm/s, beneficially 5 mm/s, enables frames to be acquired with inter-frame spacing of 0.2 mm to 0.3 mm, beneficially 0.25 mm.

An ultrasound echo signal is then received for the short-axis cross-section of the carotid artery corresponding to each position to which the ultrasound probe 2 has been displaced. Electrical signals are sequentially converted from the ultrasound echo signal and supplied to the transmission and reception processing unit 3. The above operation is hereinafter referred to as a hand scan.

(Transmission and Reception Processing Unit 3)

The transmission and reception processing unit 3 generates an electronic signal in pulse or continuous form for causing the ultrasound probe 2 to transmit an ultrasound, and performs a transmission process of providing the electronic signal to the ultrasound probe 2 as a transmission signal.

The transmission and reception processing unit 3 performs a reception process of amplifying the electronic signal received from the ultrasound probe 2 and performing analogue-to-digital (hereinafter, A/D) conversion to generate a reception signal. The reception signal is, for example, made up of a plurality of signals obtained along the transducer array direction and in a depth direction oriented away from the transducer array. Each of these is a digital signal obtained by performing A/D conversion on an electronic signal converted from the amplitude of an echo signal. Here, the reception signal is generated for the short-axis cross-section of the carotid artery in a plurality of frames corresponding to the above-described hand scan. The reception signal for each of these frames is supplied to the two-dimensional image generation unit 4.

(Two-dimensional Image Generation Unit 4)

The two-dimensional image generation unit 4 generates a two-dimensional image of a carotid artery short-axis corresponding to each frame based on the reception signal, then supplies the images to the two-dimensional image storage unit 5. Each two-dimensional image is an image signal in which a coordinate conversion has been applied to the reception signal with respect to a Cartesian coordinate system. The two-dimensional images are stored in the two-dimensional image storage unit 5 along with an ordering obtained in the hand scan.

(Bulb Boundary Detection Unit 15)

The bulb boundary detection unit 15 measures at least one of a perimeter and a cross-sectional area of the carotid artery vascular wall according to the carotid artery short-axis cross-section as seen in each of the two-dimensional images. Then, the bulb boundary detection unit 15 detects a boundary between the common carotid artery and the bulb according to said one of the perimeter and cross-sectional area, and information indicating the position in the carotid artery at which each two-dimensional image was obtained. The bulb boundary detection unit 15 includes a border extraction unit 6, a three-dimensional volume data storage unit 7, a cross-section information analysis unit 8, a cross-section analysis data storage unit 9, a vascular direction determination unit 10, a vascular direction data storage unit 11, and a bulb boundary determination unit 12. The function of each component is described below.

(Border Extraction Unit 6)

The border extraction unit 6 uses a general image processing method, such as edge detection, to extract a border of the carotid artery vascular wall based on the two-dimensional images stored in the two-dimensional image storage unit 5.

(Three-Dimensional Volume Data Storage Unit 7)

The three-dimensional volume data storage unit 7 stores the border extracted by the border extraction unit 6 for each frame. The collection of border data stored for the frames is hereinafter termed three-dimensional volume data.

(Cross-Section Information Analysis Unit 8)

The cross-section information analysis unit 8 analyses the three-dimensional volume data stored in the three-dimensional volume data storage unit 7 regarding whether or not the hand scan has been correctly performed. The hand scan is performed by transmitting and receiving an ultrasound beam during displacement one way along the skin surface in the longitudinal direction of the carotid artery. Thus, a portion of the frame data may be missing due to the manner in which the hand scan is performed. The cross-section information analysis unit 8 is provided in order to prevent data errors due to missing data.

FIG. 3 is a block diagram illustrating the configuration of a cross-section information analysis unit 8 in the ultrasound diagnostic device 1 pertaining to Embodiment 1. The cross-section information analysis unit 8 includes a vascular position detection unit 80, a vascular perimeter measurement unit 81, a vascular cross-sectional area measurement unit 82, a vascular cross-section detection unit 83, and a cross-section information determination unit 84.

The vascular position detection unit 80 detects a position of the vascular border in a single frame.

The vascular perimeter measurement unit 81 measures the length of the perimeter in the border according to position information of the vascular border obtained by the vascular position detection unit 80. The length of the perimeter is obtainable by taking any of a lumen-intima boundary perimeter, a media-adventitia boundary perimeter, and a circumference of the vascular wall, the latter being the outermost portion of the blood vessel. Given that the tunica intima and tunica media of the vascular wall are easily deformable under the influence of the pulse, and that appropriate vascular position is difficult to detect when plaque has formed in the lumen, extracting the media-adventitia boundary of the circumference of the vascular wall as the vascular wall is beneficial in that these issues have less effect.

Like the vascular perimeter measurement unit 81, the vascular cross-sectional area measurement unit 82 measures the area of the border according to position information of the border. Here, the cross-sectional area of the border is obtainable from any of a range occupied by the lumen and surrounded by the lumen-intima boundary in the carotid artery, a range surrounded by the media-adventitia boundary, and a range surrounded by the circumference of the vascular wall that is the outermost portion of the blood vessel. Given that the tunica intima and tunica media of the vascular wall are easily deformable under the influence of the pulse, and that appropriate vascular position is difficult to detect when plaque has formed in the lumen, extracting the media-adventitia boundary of the circumference of the vascular wall as the vascular wall is beneficial in that these issues have less effect.

The vascular cross-section detection unit 83 detects the number of carotid artery cross-sections in a single frame. For areas farther along the peripheral direction than the Bif 217, two carotid artery cross-sections are present in a single frame.

The cross-section information determination unit 84 determines whether or not the frames obtained in the hand scan are continuous, according to results of the above-described process, and thus checks whether or not the hand scan has been performed correctly. That is, the cross-section information determination unit 84 performs a determination process of determining whether or not the two-dimensional images have been acquired at positions along the carotid artery with the predetermined spacing. Thus, the cross-section information determination unit 84 detects a coordinate position of the carotid artery in each two-dimensional image, and determines that the two-dimensional images have been acquired at positions along the carotid artery with the predetermined spacing when an offset between coordinate positions in neighbouring two-dimensional images is equal to or less than a predetermined threshold.

The process performed by the cross-section information analysis unit 8 is applied to each of the frames in the three-dimensional volume data. Information obtained as a result of this process is supplied to and stored in the cross-section analysis data storage unit 9.

In Embodiment 1, the later-described bulb boundary determination unit 12 is able to make a highly precise determination. Thus, only one of the vascular perimeter measurement unit 81 and the vascular cross-sectional area measurement unit 82 is necessary when the determination by the bulb boundary determination unit 12 is not used.

(Vascular Direction Determination Unit 10)

The vascular direction determination unit 10 determines whether the hand scan has been performed from the CCA toward the ICA/ECA (hereinafter, forward) or from the ICA/ECA toward the CCA (hereinafter, backward), according to the data stored in the cross-section analysis data storage unit 9. That is, the direction of the hand scan is determined from a plurality of frames in the three-dimensional volume data. Specifically, variations in the border detected by the vascular position detection unit 80 with respect to the longitudinal direction are used to identify some of frames as central direction data and other frames as peripheral direction data. When the spacing between the respective coordinate positions of the front wall and the back wall increases along the longitudinal direction, that direction is the peripheral direction given that the vascular diameter gradually increases over distance from the CCA to the bulb.

(Vascular Direction Data Storage Unit 11)

The vascular direction data storage unit 11 stores information obtained by the vascular direction determination unit 10.

(Bulb Boundary Determination Unit 12)

The bulb boundary determination unit 12 detects, as the aforementioned boundary, the onset of change in the vascular perimeter or in the cross-sectional area extracted from each two-dimensional image of the frames. Specifically, the boundary between the CCA and the bulb is detected as an inflection point based on the data in the cross-section analysis data storage unit 9 and the vascular direction data storage unit 11. The details are described later.

(ROI Determination Unit 13)

The ROI determination unit 13 determines the ROI 211 defining a range for IMT measurement according to the inflection point obtained by the bulb boundary determination unit 12. That is, a frame corresponding to a predetermined IMT measurement range 212 is selected. For example, for correspondence with the recommended IMT measurement range of Non-Patent Literature 1, a frame is selected in which a range of 1 cm toward the CCA from the inflection point is visible. This is selected as the IMT measurement range 212. As described above, when the ultrasound probe is displaced at a fixed speed along the longitudinal direction of the carotid artery during ultrasound image capture, the frames corresponding to the short-axis cross-section images are acquired with fixed spacing. In such a case, each centimeter is divided by the speed of ultrasound probe displacement to obtain a number of frames to be used as the IMT acquisition target. Here, the speed of ultrasound probe displacement is beneficially a fixed predetermined speed, such as 4 mm/s to 6 mm/s.

(IMT Measurement Unit 14)

The IMT measurement unit 14 acquires a two-dimensional image from the two-dimensional image storage unit 5 corresponding to the frames selected by the ROI determination unit 13 and performs the IMT measurement on each such frame based on the corresponding two-dimensional image. As described above, the vascular wall 201 is made up of the tunica intima 202, the tunica media 203, and the tunica adventitia 205. The IMT is a thickness measurement of the intima-media 206, which is a complex of the tunica intima 202 and the tunica media 203. The IMT measurement unit 14 measures the IMT by detecting the intima-media 206 between the lumen 204 and the tunica adventitia 205 in the two-dimensional image generated according to the reception signal. The two-dimensional image is a tomogram of a blood vessel, showing the vascular cross-section in the short-axis direction. A method for measuring the IMT from such an image is described in International Patent Application Publication No. 2012/105162, for instance.

The results of IMT measurement are then displayed on a (non-diagrammed) display. Alternatively, a three-dimensional image of the carotid artery may be constructed by combining the borders in the frames obtained through the hand scan. Displaying the IMT measurement range 212 for the IMT measurement on such a three-dimensional image provides a configuration that is easier for the operator to use.

### Operations

The operations of the ultrasound diagnostic device 1 configured as described above are explained with reference to the flowchart of FIG. 4. FIG. 4 is a flowchart of operations pertaining to IMT measurement by the ultrasound diagnostic device 1 of Embodiment 1. The transmission and reception of the ultrasound beam to the subject having the carotid artery are performed by typical methods, and explanations thereof are thus omitted. That is, the following explains operations beginning when the ROI 211 is automatically determined and ending when the IMT is measured in the ROI 211.

(Step 1 (S01))

In step 1 (S01), the transmission and reception processing unit 3 generates a reception signal for each frame, based on the ultrasound echo signal obtained by the ultrasound probe 2 during the hand scan. In order to acquire a two-dimensional image of the carotid artery short-axis cross-section, the ultrasound probe 2 is arranged such that the transducer array is substantially perpendicular to the longitudinal direction of the carotid artery. the ultrasound probe 2 is then displaced along the longitudinal direction of the carotid artery. In order to obtain a plurality of two-dimensional images of the short-axis cross-section in a predetermined interval, the displacement of the ultrasound probe 2 is beneficially a motion along the longitudinal direction of the carotid artery performed at a predetermined fixed speed.

(Step 2 (S02))

FIGS. 5A-5C illustrate data based on the two-dimensional image indicating a short-axis cross section of the carotid artery as obtained by the ultrasound diagnostic device 1 of Embodiment 1. FIG. 5A illustrates the configuration of a two-dimensional image 100, FIG. 5B illustrates the configuration of a carotid artery border 101 in each frame, and FIG. 5C illustrates the configuration of a three-dimensional image 102 of the carotid artery.

In step 2 (S02), the two-dimensional image storage unit 5 generates a two-dimensional image 100 for each frame as illustrated in FIG. 5A, in accordance with the reception signal obtained in step 1. The two-dimensional image 100 so generated is then stored in the two-dimensional image storage unit 5.

(Step 3 (S03))

In step (S03), a typical image processing method such as that described above is applied to the two-dimensional image 100 stored in two-dimensional image storage unit 5 to extract a carotid artery border 101. The border 101 in each frame is stored as three-dimensional volume data in the three-dimensional volume data storage unit 7. Next, the border 101 in each of the frames obtained in the hand scan are connected to construct a three-dimensional image 102 of the carotid artery as shown in FIG. 5C. Next, the three-dimensional image 102 is stored in the three-dimensional volume data storage unit 7.

(Step 4 (S04))

In step 4 (S04), a determination is made regarding whether or not the three-dimensional volume data are from a correctly-performed hand scan. FIG. 6 is a flowchart of the operations performed by the ultrasound diagnostic device 1 of Embodiment 1 to determine whether or not the hand scan has been correctly performed. Step 4 (S04) is described with reference to FIG. 6.

(Step 41 (S41))

In step 41 (S41), the vascular position detection unit 80 scans the two-dimensional images subjected to image processing by the border extraction unit 6, and detects a coordinate position of the border 101 as X-Y coordinates indicating the border 101 in FIG. 5B. Here, borders other than the carotid artery vascular wall may appear as noise. This noise may be treated by, for example, detecting a non-loop border as not being the carotid artery cross-section and ignoring or deleting that noise.

(Step 42 (S42))

In step 42 (S42), the vascular perimeter measurement unit 81 measures the perimeter of the border 101 according to the coordinate position detected for the border 101 by the vascular position detection unit 80. This represents the carotid artery perimeter. The border perimeter is usable as the perimeter of the carotid artery tunica adventitia outer diameter.

(Step 43 (S43))

In step 43 (S43), the vascular cross-sectional area measurement unit 82 measures the cross section of the border 101 according to the coordinate position detected for the border 101 by the vascular position detection unit 80. This represents the carotid artery cross-sectional area. The cross-sectional area of the border is used as a range surrounding the outer diameter of the carotid artery tunica adventitia, which includes the area taken up by the lumen.

Steps 42 (S42) and 43 (S43) may also be performed in the opposite order, such that step 42 (S42) follows step 43 (S43). Also, as described above, the configuration may also be such that only one of the perimeter and cross-sectional area of the carotid artery is measured. In such a case, one of these steps is not performed.

When measuring the perimeter and cross-sectional area of the carotid artery, the positions of the lumen-intima boundary and the media-adventitia boundary, as well as the outer perimeter of the tunica adventitia may be used as reference for measurement. However, the tunica intima and tunica media of the vascular wall are easily deformable under the influence of the pulse. Also, appropriate measurement of the perimeter and cross-sectional area is difficult when plaque has formed in the lumen. As such, the perimeter and cross-sectional area of the carotid artery are beneficially measured with reference to the position of the media-adventitia boundary or the outer perimeter of the tunica adventitia, which are less affected by these issues.

(Step 44 (S44))

In step 44 (S44), the number of carotid artery cross-sections in each frame is detected. Normally, the number of carotid artery cross-sections obtained by the above-described process is one for a frame of the CCA and two for a frame of the ICA and ECA, which are farther along the peripheral direction than the bulb. Accordingly, the number of cross-sections can never be three or greater. However, the vascular position detection unit 80 may detect unprocessed loops of the border 101. Such loops are identified as noise using the information obtained by the vascular perimeter measurement unit 81 or the vascular cross-sectional area measurement unit 82, and the noise may be ignored or deleted. Specifically, the carotid artery has a diameter on the order of 10 mm and is very large in comparison to typical blood vessels. As such, numerical information such as the perimeter and the cross-sectional area serves as a basis for distinguishing the carotid artery cross-section from noise.

(Step 45 (S45))

In step 45 (S45), the cross-section information determination unit 84 uses the processing of steps 41 (S41) through 44 (S44) to determine whether or not the frames obtained in the hand scan are continuous.

Specifically, for each frame, a determination is made based on the X-Y coordinate position of the border 101 identified as the carotid artery cross-section and the X-Y coordinate position of the border 101 corresponding to an immediately-preceding frame from the hand scan. For example, the central position of the border 101 in each frame is compared, and when the difference therebetween is equal to or less than a predetermined threshold, the determination is such that continuous frames have been obtained and that the hand scan has been performed correctly. In such an affirmative case, the data are stored in the cross-section analysis data storage unit 9 and the process advances to step 5 (S05). However, in the negative case, the hand scan is redone without proceeding to the next step.

(Step 5 (S05))

In step 5 (S05), the vascular direction determination unit 10 determines the direction of the hand scan and stores that direction in the vascular direction data storage unit 11. FIG. 7A schematically illustrates the carotid artery when the hand scan is performed forward (in the direction of the arrow), and FIG. 7B schematically shows two-dimensional image as cross-sectional diagrams (A) through (D) of the carotid artery as obtained by the scan of FIG. 7A.

The two-dimensional images of FIG. 7B include image (A) of the CCA 213, image (B) of the vicinity of the boundary between the CCA 213 and the bulb 214, image (C) of the vicinity of the Bif 217, and image (D) of the ICA 215 and the ECA 216. As shown in FIG. 7b, image (A) includes one border 101 corresponding to the carotid artery cross-section, and is thus identifiable as the CCA 213 side. Conversely, image (D) includes two borders, and is thus identifiable as the ICA 215 and ECA 216 side. Given that these two-dimensional image have been acquired in order beginning with image (A), the hand scan is identifiable as having been performed in the forward direction.

(Step 6 (S06))

In step 6 (S06), the bulb boundary determination unit 12 detects the boundary between the CCA and the bulb is detected as the inflection point based on the data in the cross-section analysis data storage unit 9 and the vascular direction data storage unit 11. The inflection point at the boundary between the CCA and the bulb is detected by associating the perimeter and cross-sectional area of the border 101 with each respective frame of the three-dimensional volume data, taking the second derivative of the perimeter and cross-sectional area, and extracting the inflection point of the perimeter and cross-sectional area values.

First, the perimeter and cross-sectional area of the border 101 in each frame of the three-dimensional volume data are associated with each frame. FIG. 8 lists frame numbers in the reception signal obtained by the ultrasound diagnostic device 1 of Embodiment 1, and the perimeter and cross-sectional area for the border 101 in each frame. The first frame is labelled frame 0, followed by frames 1, 2, . . . n.

Next, the perimeter and cross-sectional area of the border 101 corresponding to each frame in the three-dimensional volume data shown in FIG. 8 are plotted in order of acquisition during the hand scan. FIG. 9 shows a plotted relationship frame numbers in the reception signal obtained by the ultrasound diagnostic device 1 of Embodiment 1, and the perimeter and cross-sectional area for the border 101 associated with each frame, in hand scan order. In FIG. 9, the area is calculated as a number of pixels in the vascular cross-section as shown in the two-dimensional image, and the perimeter is calculated a number of pixels making up the boundary between the vascular cross-section as shown in the two-dimensional image and other portions of the image. Data for the CCA and the bulb is sufficient for detecting the inflection point. ICA and ECA data are not required. Accordingly, in Embodiment 1, the ICA and ECA data are omitted, and only the data for the CCA and bulb are shown.

As shown in FIG. 9, the perimeter and cross-sectional area are subject to nearly identical variation. In the CCA, the perimeter and cross-sectional area extend with nearly constant values.

As described with reference to FIG. 18B, the round shape of the bulb produces a increase in vascular diameter at the transition from the CCA-bulb boundary into the bulb itself. As a result, a sudden increase in perimeter and cross-sectional area also occurs at the transition from the CCA-bulb boundary into bulb. The data given in FIG. 9 also indicate that the vascular perimeter and cross-sectional area in each frame nearly match in terms of variation. The inflection point at the boundary between the CCA and the bulb corresponds to the onset of change from the stable CCA portion, in terms of the perimeter and cross-sectional area. As such, the onset of increasing values for the perimeter and cross-sectional area, as shown in FIG. 9, is detected as the inflection point.

However, the onset of change in the perimeter and cross-sectional area (i.e., the inflection point) is not easy to find in the data shown in FIG. 9. Accordingly, a calculation such as averaging the data in FIG. 9 and taking the second derivative may be performed to more easily detect the onset of change (i.e., the inflection point).

FIG. 10 is a plotted relationship between the frame numbers and the results of taking a moving average from the last two instances and computing the second derivative in each frame, in hand scan order, from the cross-sectional area (the data of FIG. 9) of the border 101 in each frame acquired from the reception signal obtained by the ultrasound diagnostic device 1 of Embodiment 1. The inflection point at the boundary between the CCA and the bulb is found, in FIG. 10, to be at frame 135, as seen upon applying the second derivative calculation process.

(Step 7 (S07))

In step 7 (S07), the IMT measurement range 212 is determined from the bulb boundary at the inflection point detected in step 6 (S06). Specifically, the ROI determination unit 13 determines the ROI 211 defining a range for IMT measurement according to the inflection point obtained by the bulb boundary determination unit 12. That is, a frame corresponding to a predetermined IMT measurement range 212 is selected. For example, for correspondence with the recommended IMT measurement range of Non-Patent Literature 1, a frame is selected in which a range of 1 cm toward the CCA from the inflection point is visible. This is selected as the IMT measurement range 212.

(Step 8 (S08))

In step 8 (S08), the IMT is measured in the determined IMT measurement range 212. Specifically, the IMT measurement unit 14 performs the IMT measurement on each frame selected by the ROI determination unit 13 based on the corresponding two-dimensional image in the from the two-dimensional image storage unit 5. For example, IMT measurement is performed on each frame corresponding to the IMT measurement range 212, and an IMT value is confirmed by using measurement results such as the maxIMT or meanIMT.

### Effects

According to the above configuration, the ultrasound diagnostic device 2 of Embodiment 1 is able to detect the CCA-bulb boundary 219 using the perimeter and cross-sectional area obtained from a short-axis cross-section image and without depending on longitudinal direction curvature, despite, for example, a two-dimensional image being acquired in which great curvature in the longitudinal direction is present from the CCA to the bulb. Also there is no need to place the transducer array near the centre of the short-axis cross-section, such as when measuring IMT with a longitudinal direction cross-section image. According to this configuration, the CCA-bulb boundary 219 is easily detectable irrespective of the subject's carotid artery shape, and the CCA-bulb boundary 219 is also automatically detectable. Thus, the ultrasound diagnostic device 1 performing an IMT measurement of a carotid artery vascular wall automatically determines the ROI 211 defining the measurement range for such measurement, enabling swift IMT measurement that is convenient for an inexperienced operator.

### Variation

Although the ultrasound diagnostic device has been described according to the above Embodiments, no such limitation is intended. The following variations may also be applied to the ultrasound diagnostic device.

In Embodiment 1, the IMT measurement unit 14 performs the IMT measurement according to a two-dimensional image. However, IMT measurement may also be performed be performed according to a reception signal for each frame, received by the transmission and reception processing unit 3. FIG. 11 is a block diagram illustrating the configuration of an ultrasound diagnostic device 1′ pertaining to a variation of Embodiment 1. A reception signal storage unit 50 is positioned after the transmission and reception processing unit 3, and stores the reception signal supplied by the transmission and reception processing unit 3. The IMT measurement unit 14 acquires a reception signal from the reception signal storage unit 50 corresponding to the frames selected by the ROI determination unit 13 and performs the IMT measurement on each such frame based on the corresponding reception signal. As described above, the vascular wall 201 is made up of the tunica intima 202, the tunica media 203, and the tunica adventitia 205. The IMT is a thickness measurement of the intima-media 206, which is a complex of the tunica intima 202 and the tunica media 203. The IMT measurement unit 14 measures the IMT by detecting the intima-media 206 between the lumen 204 and tunica adventitia 205 in the reception signal. The results of IMT measurement are then displayed on a (non-diagrammed) display.

### Embodiment 2

The ultrasound diagnostic device 1 of Embodiment 1 analyses a cross-sectional image taken along the short axis of the carotid artery as obtained by a hand scan with the ultrasound probe 2 in the longitudinal direction of the carotid artery, extracts the CCA-bulb boundary 219, and determines the ROI 211 defining a measurement range for performing IMT measurement. A ultrasound diagnostic device 1A pertaining to Embodiment 2 uses a ultrasound probe 2A in which the transducer array oscillates in a direction perpendicular to itself. Once the transducer array is arranged along the longitudinal direction of the carotid artery, two-dimensional images of the carotid artery are respectively acquired in the short-axis and long-axis directions. The two-dimensional images are analysed to extract a CCA-bulb boundary 219 and determine a ROI 211 defining a range for IMT measurement.

FIG. 12 is a perspective view diagram illustrating the configuration of an ultrasound diagnostic device 1A pertaining to Embodiment 2. As shown, the ultrasound diagnostic device 1A of Embodiment 2 has the transducer array of the ultrasound probe 2A arranged along the longitudinal direction carotid artery and acquires respective short-axis and long-axis two-dimensional images of the carotid artery to perform IMT measurement. The ultrasound probe 2A has transducers that oscillate in a direction perpendicular to the array direction. A plurality of short-axis cross-section two-dimensional images is obtained in correspondence to the pixels of the transducers that oscillate. Also, a two-dimensional image in the longitudinal cross-section of the carotid artery is acquired at each oscillation position. The ROI 211 defining the measurement range for measuring the IMT is then determined according to the two-dimensional short-axis cross-section images. The IMT is then measured from a two-dimensional image in the longitudinal direction, where the ROI 211 is found.

### Configuration

(General Configuration)

The specific configuration of the ultrasound diagnostic device 1A pertaining to Embodiment 2 is described below. FIG. 13 is a block diagram illustrating the configuration of an ultrasound diagnostic device 1A pertaining to Embodiment 2. The ultrasound diagnostic device 1A of Embodiment 2 includes a transmission and reception processing unit 3A, a two-dimensional image generation unit 4A, a two-dimensional image storage unit 5A, and an IMT measurement unit 14A. These differ from the corresponding components of the ultrasound diagnostic device 1 of Embodiment 1. Other components are configured identically to those shown in FIG. 1, and explanations thereof are thus omitted.

(Ultrasound Probe 2A)

The ultrasound diagnostic device 1A of Embodiment 2 is configured to be electrically connectable to the ultrasound probe 2A. The ultrasound probe 2A has transducers arranged in a row aligning with the longitudinal direction of the carotid artery. An ultrasound beam is transmitted along the longitudinal direction of the carotid artery and an ultrasound echo signal is received as a reflected ultrasound. Accordingly, the ultrasound echo signal is received for generating the two-dimensional image of the carotid artery in longitudinal cross-section. Also, as shown in FIG. 12, when the transducers of the ultrasound probe 2A oscillate perpendicularly with respect to the array direction, an ultrasound echo signal is acquired for generating the short-axis cross-section two-dimensional image of the carotid artery. Further, an ultrasound echo signal for the two-dimensional image in the longitudinal cross-section of the carotid artery is acquired at each oscillation position.

(Transmission and Reception Processing Unit 3A)

The transmission process performed by the transmission and reception processing unit 3A is as described in Embodiment 1. However, the reception process includes generating a reception signal for generating a short-axis cross-section two-dimensional image of the carotid artery for each frame, and generating the reception signal for generating a longitudinal cross-section of the carotid artery at each oscillation position.

(Two-dimensional Image Generation Unit 4A)

The two-dimensional image generation unit 4 uses the reception signal from the transmission and reception processing unit 3A to generate the short-axis cross-section two-dimensional image of the carotid artery for each frame, and to generate the longitudinal cross-section two-dimensional image for the carotid artery at each oscillation position. Among the longitudinal cross-section two-dimensional image of the carotid artery so acquired, the two-dimensional image used for IMT measurement is, for example, obtained at a central position during oscillation, or acquired at a predetermined oscillation position while passing the centre of the carotid artery.

(Two-Dimensional Image Storage Unit 5A)

The two-dimensional image storage unit 5A stores the longitudinal cross-section two-dimensional image of the carotid artery for each oscillation position. The stored two-dimensional images are used for IMT measurement. As in Embodiment 1, the short-axis cross-section two-dimensional images of the carotid artery for each frame are supplied to the border extraction unit 6.

Afterward, the bulb boundary detection unit 15 applies the process described in Embodiment 1 to the short-axis cross-section two-dimensional image to detect the boundary between the CCA and the bulb. The ROI determination unit 13 then applied the process described in Embodiment 1 to determine the ROI 211 that defines the measurement range for measuring the IMT at the CCA-bulb boundary.

(IMT Measurement Unit 14A)

The IMT measurement unit 14A measures the IMT in the two-dimensional image having the ROI 211 determined by the determination unit, the two-dimensional image being of the carotid artery longitudinal cross-section. A method for measuring the IMT from the cross-sectional image in the longitudinal direction is described in International Patent Application Publication No. 2007/108359, for instance. The two-dimensional image generated by the two-dimensional image generation unit 4A and used for IMT measurement is beneficially obtained at a central position during oscillation, or acquired at a predetermined oscillation position passing the centre of the carotid artery, for example. As described above, in order to realise an accurate IMT measurement, the measurement must be performed when the transducer array is in the vicinity of the short-axis cross-section centre along the longitudinal direction of the carotid artery, as shown in FIG. 17A.

### Effects

According to the above configuration, the ultrasound diagnostic device 1A pertaining to Embodiment 2, like the ultrasound diagnostic device 1 pertaining to Embodiment 1, detects the CCA-bulb boundary 219 from the cross-sectional area and perimeter obtained from the short-axis cross section of the artery and determines the ROI 211 accordingly, given that these measurements are independent of longitudinal direction curvature. Also, the oscillation of the ultrasound probe 2 enables IMT measurement in an ROI 211 from a two-dimensional image of the longitudinal direction obtained when the ultrasound probe 2 is in the vicinity of the artery centre.

According to this configuration, the CCA-bulb boundary 219 is easily detectable irrespective of the subject's carotid artery shape, and the CCA-bulb boundary 219 is also automatically detectable. Thus, the ultrasound diagnostic device 1 performing an IMT measurement of a carotid artery vascular wall automatically determines the ROI 211 defining the measurement range for such measurement, enabling swift IMT measurement that is convenient for an inexperienced operator.

### Variation

Although the ultrasound diagnostic device 1A of Embodiment 2 has been described above, no limitation thereto is intended. The following variations may also be applied to the ultrasound diagnostic device.

The IMT measurement unit 14A of Embodiment 2 measures the IMT in the two-dimensional image having the ROI 211 determined by the determination unit, the two-dimensional image being of the carotid artery longitudinal cross-section. However, like the IMT measurement unit 14 of Embodiment 1, the two-dimensional image in the short-axis direction corresponding to a plurality of frames that include the ROI 211 selected by the ROI determination unit 13 may be acquired from the two-dimensional image storage unit 5, and the IMT measurement may be performed in each frame based on the two-dimensional image. A method for measuring the IMT from the cross-sectional image in the short-axis direction is described in International Patent Application Publication No. 2012/105162, for instance. Like the ultrasound diagnostic device 1A pertaining to Embodiment 2, determining the ROI 211 according to the boundary detected by the CCA-bulb boundary 219 enables IMT measurement to be performed from the two-dimensional images of a short-axis cross-section, unaffected by offset of the transducer array from the artery centre.

### CONCLUSION

According to the above, Embodiments 1 and 2 detect the CCA-bulb boundary according to longitudinal direction variation in the cross-sectional area and perimeter found in a short-axis cross section of the carotid artery, and determine the ROI 211 defining the range for IMT measurement according to the CCA-bulb boundary. Thus, the ultrasound diagnostic device performing an IMT measurement of a carotid artery vascular wall automatically determines the ROI 211 defining the measurement range for such measurement, enabling swift IMT measurement that is convenient for an inexperienced operator.

### Supplement

Each of the Embodiments described above is a non-limiting Embodiment of the device of the present disclosure. The quantities, shapes, materials, components, arrangement position and connections between components, steps, order of steps, and so on listed in the Embodiments are given as examples, no limitation being intended thereby. Also, the components listed in the Embodiments and steps not listed as independent aspects representing highest-order concepts of the disclosure are described as optional components for a beneficial Embodiment.

Furthermore, in drawings referred to in the above Embodiments, in order to facilitate understanding, configuration elements are not necessarily illustrated to scale. The present disclosure is not limited by the above Embodiments, and appropriate modifications may be made so long as such modifications do not cause deviation from the general concept of the present disclosure.

Furthermore, the ultrasound diagnostic device is configured as circuit elements, lead lines, and so on disposed on a substrate. However, the electrical connections and circuits thereof are technological matter widely known from ultrasound diagnostic apparatus technology and the like and are applicable to various configurations. As such, explanations thereof are omitted as not being directly relevant to the disclosure. The above-described drawings are schematics that do not necessarily closely conform to reality.

### INDUSTRIAL APPLICABILITY

The present disclosure is widely applicable to an ultrasound diagnostic device and an ultrasound diagnostic device control method enabling automatic determination of a ROI 211 for defining a measurement range in which to measure IMT of a carotid artery vascular wall, and enabling IMT measurement to be quickly performed by an inexperienced operator through simple operations.

### LIST OF REFERENCE SIGNS

1, 1′, 1A Ultrasound diagnostic device
2, 2A Ultrasound probe
3, 3A Transmission and reception processing unit
4, 4A Two-dimensional image generation unit
5, 5A Two-dimensional image storage unit
6 Border extraction unit
7 Three-dimensional volume data storage unit
8 Cross-section information analysis unit
9 Cross-section analysis data storage unit
10 Vascular direction determination unit
11 Vascular direction data storage unit
12 Bulb boundary determination unit
13 ROI determination unit
14, 14A IMT measurement unit
15 Bulb boundary detection unit
50 Reception signal storage unit
80 Vascular position detection unit
81 Vascular perimeter measurement unit
82 Vascular cross-sectional area measurement unit
83 Vascular cross-section detection unit
84 Cross-section information determination unit
100 Two-dimensional image
101 Border
102 Three-dimensional image
201 Vascular wall
202 Tunica intima
203 Tunica media
204 Lumen
205 Tunica adventitia
206 Intima-media
207 Lumen-Intima boundary
208 Media-Adventitia boundary
209 Back wall
210 Front wall
211 Region of interest (ROI)
212 IMT measurement range
213 Common Carotid Artery (CCA)
214 Bulb of the common carotid artery (Bulb)
215 Internal Carotid Artery (ICA)
216 External Carotid Artery (ECA)
217 Bifurcation of the common carotid artery (Bif)
219 CCA-Bulb boundary
220 Artery center
300, 300A Controller

1. An ultrasound diagnostic device to which an ultrasound probe is connectable, measuring an IMT of a vascular wall in a carotid artery, comprising:
a transmission and reception processing unit performing a transmission process of supplying a transmission signal for causing the ultrasound probe to transmit an ultrasound to a short-axis cross-section at a plurality of different positions along the carotid artery, and a reception process of receiving a signal based on a reflected ultrasound received by the ultrasound probe from the carotid artery and generating a reception signal for a plurality of frames each corresponding to the short-axis cross-section at one of the different positions;
a two-dimensional image generation unit generating a plurality of two-dimensional images each corresponding to one of the frames, based on the reception signal for each of the frames;
a bulb boundary detection unit measuring one or more of a perimeter and a cross-sectional area of the vascular wall in the carotid artery according to the short-axis cross-section in each of the two-dimensional images, and detecting a boundary between a common carotid artery and a common carotid artery bulb according to: at least one of the perimeter and the cross-sectional area; and position information indicating a respective position along the carotid artery where each of the two-dimensional images is acquired;
a ROI determination unit determining a ROI that defines a measurement range for measuring the IMT, with respect to the boundary; and
an IMT measurement unit measuring the IMT in at least one of the two-dimensional images in the ROI.

2. The ultrasound diagnostic device of claim 1, wherein
the bulb boundary detection unit detects the boundary as an onset of change in size of the perimeter or of the cross-sectional area.

3. The ultrasound diagnostic device of claim 1, wherein
the bulb boundary detection unit performs a border extraction process of extracting a border of the vascular wall in the carotid artery from the respective two-dimensional images corresponding to each of the frames, and measures one or more of the perimeter and the cross-sectional area using the border.

4. The ultrasound diagnostic device of claim 1, wherein
the bulb boundary detection unit performs a determination process of determining whether acquisition of the two-dimensional images has been performed at respective positions that are separated by a predetermined interval with respect to the carotid artery.

5. The ultrasound diagnostic device of claim 4, wherein
the bulb boundary detection unit detects a coordinate position of the carotid artery for each of the two-dimensional images, and determines affirmatively when neighboring two-dimensional images are separated by a gap equal to or less than a predetermined value in terms of the respective coordinate position of the two-dimensional images.

6. The ultrasound diagnostic device of claim 1, wherein
the bulb boundary detection unit determines an extension direction of the carotid artery based on the two-dimensional images.

7. The ultrasound diagnostic device of claim 6, wherein
the bulb boundary detection unit detects a number of cross-sections present in the two-dimensional images and determines the extension direction according to the number of cross-sections and the position information.

8. The ultrasound diagnostic device of claim 6, wherein
the extension direction is one of:
a peripheral direction from the common carotid artery toward an internal carotid artery and an external carotid artery; and
a central direction from the internal carotid artery and the external carotid artery toward the common carotid artery.

9. The ultrasound diagnostic device of claim 6, wherein
the ROI determination unit determines the ROI for measuring the IMT according to the boundary and the extension direction.

10. A control method for an ultrasound diagnostic device to which an ultrasound probe is connectable, measuring an IMT of a vascular wall in a carotid artery, comprising:
supplying a transmission signal for causing the ultrasound probe to transmit an ultrasound to a short-axis cross-section at a plurality of different positions along the carotid artery;
receiving a signal based on a reflected ultrasound received by the ultrasound probe from the carotid artery and generating a reception signal for a plurality of frames each corresponding to the short-axis cross-section at one of the different positions;
generating a plurality of two-dimensional images each corresponding to one of the frames, based on the reception signal for each of the frames;
measuring one or more of a perimeter and a cross-sectional area of the vascular wall in the carotid artery according to the short-axis cross-section in each of the two-dimensional images, and detecting a boundary between a common carotid artery and a common carotid artery bulb according to: at least one of the perimeter and the cross-sectional area; and position information indicating a respective position along the carotid artery where each of the two-dimensional images is acquired;
determining a ROI that defines a measurement range for measuring a predetermined IMT, with respect to the boundary; and
measuring the IMT in at least one of the two-dimensional images in the ROI.
